(19) United States
(12) Patent Application Publication
(10) Pub. No.: US 2022/0160459 A1
(43) Pub. Date: May 26, 2022

(54) Cleaning Apparatus For Use In Machine Washing Of Surgical Handpieces

(71) Applicant: James Keith Gilbert, Williamsport, PA (US)

(72) Inventor: James Keith Gilbert, Williamsport, PA (US)

(21) Appl. No.: 17/534,584

(22) Filed: Nov. 24, 2021

Related U.S. Application Data
(60) Provisional application No. 63/117,511, filed on Nov. 24, 2020.

(51) Int. Cl. A61B 90/70; B08B 9/023; B08B 9/032

(52) U.S. Cl. CPC A61B 90/70; B08B 9/023; B08B 2209/032; A61B 2090/701; B08B 9/0321

(57) ABSTRACT

A directed flow apparatus is proposed for improving the mechanical washing of surgical instrument handpieces. The apparatus takes the form of a cleaning adaptor that is designed to attach to the distal opening of the handpiece and an inlet port connector inserted on a fluid jet of the mechanical washer. A suitable length of tubing is connected between the cleaning adaptor and the inlet port connector so that when the washing cycle begins, a high force stream of cleaning fluid is specifically directed into the interior of the handpiece and focusing the direction of the cleaning fluid into a channel that is not easily accessible otherwise.

### CROSS-REFERENCE TO RELATED APPLICATIONS

This application claims the benefit of U.S. Provisional Application No. 63/117,511, filed Nov. 24, 2020 and herein incorporated by reference.

### TECHNICAL FIELD

The present invention is directed to the field of surgical instruments and, more particularly, to cleaning apparatus useful in the machine washing of surgical handpieces.

### BACKGROUND OF THE INVENTION

Thorough cleaning of surgical instrumentation handpieces is difficult, and requires more attention than cleaning of less-complex instruments. Handpieces are precision, turbine-driven instruments that have both air and water flowing through them during a given surgical procedure. In normal use, handpieces are in direct contact with internal body tissues, and as a result may collect body fluids and tissue within its inner surfaces, as well as contamination of the mechanism of the handpiece itself. Additionally, a shaver handpiece is likely to be contaminated with miniscule fragments of bone that may be removed during an arthroscopic/orthopedic procedure.

If the debris is not removed, it may spray out of the handpiece and into a patient during a following procedure. To avoid this, the medical instrumentation industry has developed strict procedures for cleaning, sanitizing, and re-packaging handpieces for later use. A preferred procedure involves an initial manual inspection and cleaning to remove gross debris, with various types of brushes and cleaning tools used in this step. Once all visible debris is thought to be gone, the handpiece (and various other tools and instruments) are loaded into a mechanical washer for a deep cleaning. Similar to a dishwasher, but with extremely higher levels of turbulence and temperature, mechanical washing is able to further eliminate debris that cannot be seen by the naked eye (or was perhaps not found by the individual performing the manual cleaning).

A typical process for preparing a surgical instrument handpiece for mechanical washing involves loading the handpiece in a retaining basket, with the handpiece positioned at an angle so that water/cleaning fluid entering its internal passages will naturally drain through. While the use of an inclined position increases the likelihood that a fluid will enter the interior channels, this outcome cannot be presumed. Thus, the possibility remains that debris may still be resident within the handpiece at the completion of the mechanical washing procedure.

### SUMMARY OF THE INVENTION

The need remaining in the art is addressed by the present invention, which relates to a cleaning apparatus for use in conjunction with mechanical washing and, more particularly, to a directed flow apparatus that is coupled to a surgical instrumentation handpiece in a manner that intentionally directs cleaning fluid into the interior channels of the handpiece.

In accordance with one or more embodiments of the present invention, a directed flow apparatus is proposed that takes the form of a cleaning adaptor that is designed to attach to the distal opening of the handpiece and an inlet port connector inserted on a fluid jet of the mechanical washer. A suitable length of tubing is connected between the cleaning adaptor and the inlet port connector so that when the washing cycle begins, a high force stream of cleaning fluid is specifically directed into the interior of the handpiece and focusing the direction of the cleaning fluid into a channel that is not easily accessible otherwise.

An exemplary embodiment of the present invention may be defined as an apparatus for directing cleaning fluid through a medical instrumentation handpiece during a mechanical washing procedure, the apparatus comprising: (1) a cleaning adaptor configured to couple to a distal opening of a handpiece, the cleaning adaptor including a central opening for directing movement of cleaning fluid through an interior portion of the handpiece; (2) an inlet port connector configured to attach to a fluid jet outlet of a mechanical washer; and (3) a tubing element connected between the inlet port and the cleaning adaptor, providing a path for cleaning fluid exiting the mechanical washer fluid jet outlet to be directed into an interior portion of the handpiece.

Other and further aspects and embodiments of the present invention will become apparent during the course of the following discussion and by reference to the accompanying drawings.

### BRIEF DESCRIPTION OF THE DRAWINGS

Referring now to the drawings, where like numerals represent like parts in several views:

FIG. 1 illustrates an exemplary directed flow apparatus formed in accordance with the principles of the present invention;

FIG. 2 is an enlarged view of the cleaning adaptor portion of the directed flow apparatus of FIG. 1;

FIG. 3 is another view of the inventive directed flow apparatus, showing the central aperture within the cleaning adaptor portion, as well as a machine's fluid jet port attached to the opposing cleaning adaptor;

FIG. 4 is an enlarged view of the inlet connector portion of the inventive directed flow apparatus;

FIG. 5 shows the inventive directed flow apparatus in combination with a conventional handpiece, showing alignment markings that may be used to properly attaching the directed flow apparatus to the handpiece;

FIG. 6 illustrates the same combination as in FIG. 4, in this case after the directed flow apparatus has been fixed in place on the distal end of the handpiece; and

FIG. 7 illustrates an exemplary mechanical washer environment within which the inventive directed flow apparatus may be used.

### DETAILED DESCRIPTION

As mentioned above, the present invention is directed to a cleaning apparatus for use in conjunction with mechanical washing of surgical hand tools (which may be referred to hereinafter as a “surgical handpiece”) and, more particularly, to a directed flow apparatus that is coupled to the handpiece in a manner that intentionally directs cleaning fluid into the interior channels of the handpiece.

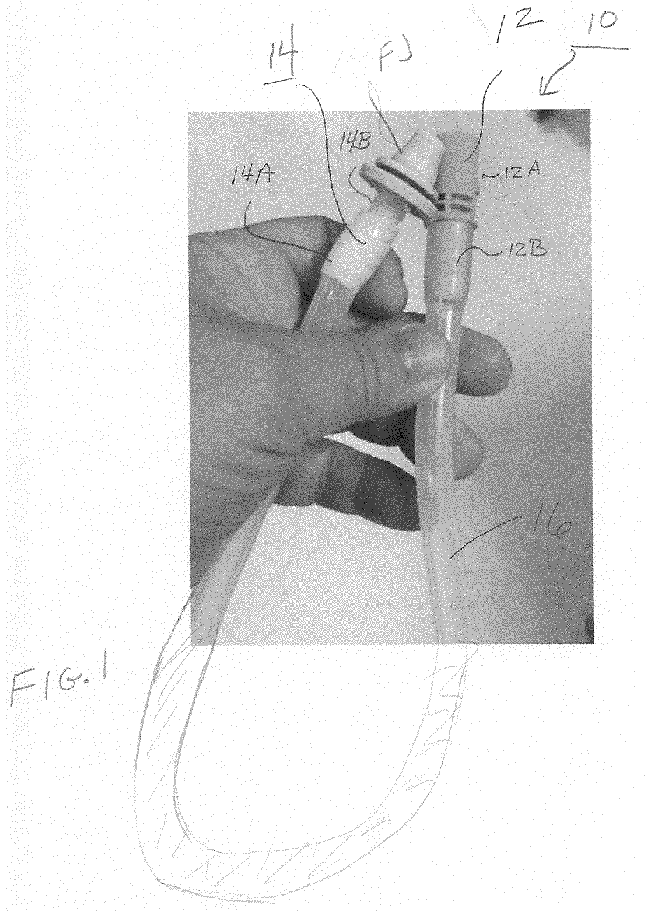

FIG. 1 illustrates an exemplary directed flow apparatus 10 formed in accordance with the principles of the present invention for this purpose. As shown, directed flow apparatus 10 comprises a cleaning adaptor 12 that is designed to attach to the distal opening of the handpiece being cleaned (not shown). Directed flow apparatus 10 further comprises an inlet port connector 14 that is specifically configured to couple to a fluid jet FJ of the associated mechanical washer apparatus. A suitable length of tubing 16 is used to provide a fluid channel for a directed flow from connector 14 through cleaning adaptor 12 and thereafter into the interior portions of the attached handpiece. Similar to cleaning adaptor 12, inlet port connector 14 comprises an apertured component (preferably with a barbed exterior) that fits down into an end section of tubing 16, with the opposing end of inlet port connector 14 configured to mate with a conventional fluid jet FJ as found in commercial washer apparatus.

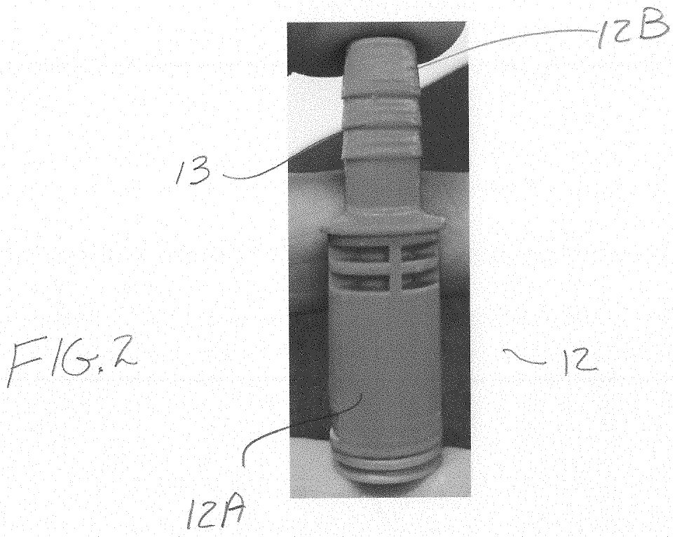

FIG. 2 is an enlarged illustration of an exemplary cleaning adaptor 12, showing a first section 12A that is specifically configured to engage with a handpiece (as will be described in detail below) and a second, contiguous section 12B that includes a tapered, apertured configuration for insertion into tubing 16. The inclusion of a fluted periphery 13 around second section 12B is considered useful in providing a secure and sealed fitting within tubing 16.

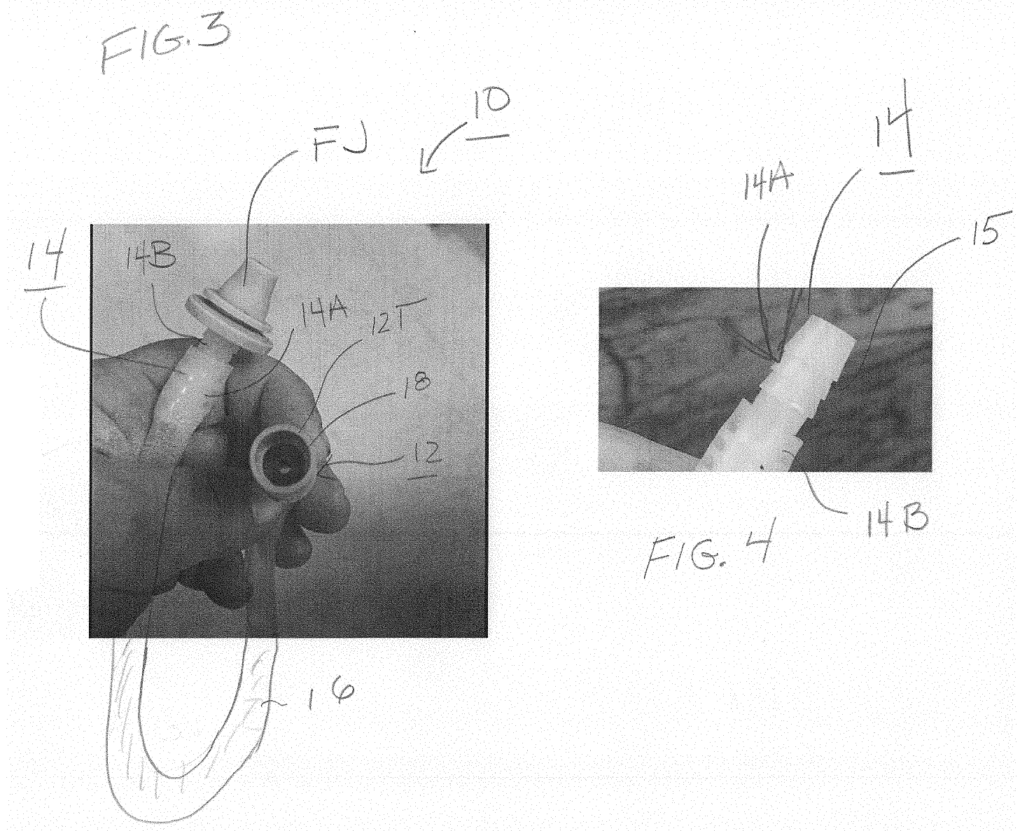

FIG. 3 is another view of directed flow apparatus 10, in this case where cleaning adaptor 12 is positioned to provide an end view of first section 12A (i.e., a top surface 12T of cleaning adaptor 12). Clearly shown in this view is an aperture 18 that comprises the fluid channel used to direct the cleaning fluid/water into the attached handpiece. Also evident in this view is the positioning of inlet port connector 14 within an opposing end section of tubing 16 (identified as interior section 14A) and an opposing exterior section 14B that mates with a standard fluid jet port FJ.

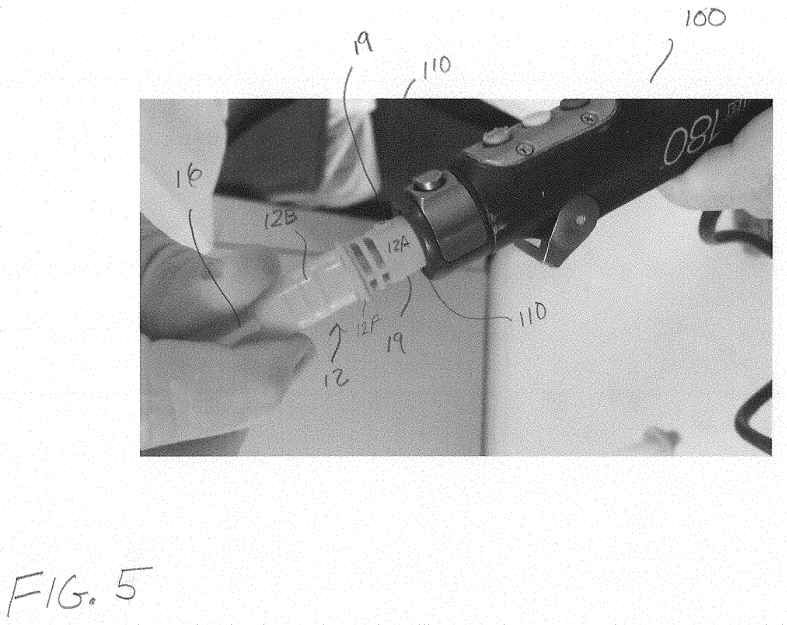

FIG. 4 is an enlarged view of an exemplary inlet port connector 14, without port FJ in place. Clearly shown in this view is a barbed outer surface 15 on interior section that enables a watertight fit of inlet port connector 14 within the end termination of tubing 16 (as also shown in FIG. 3).

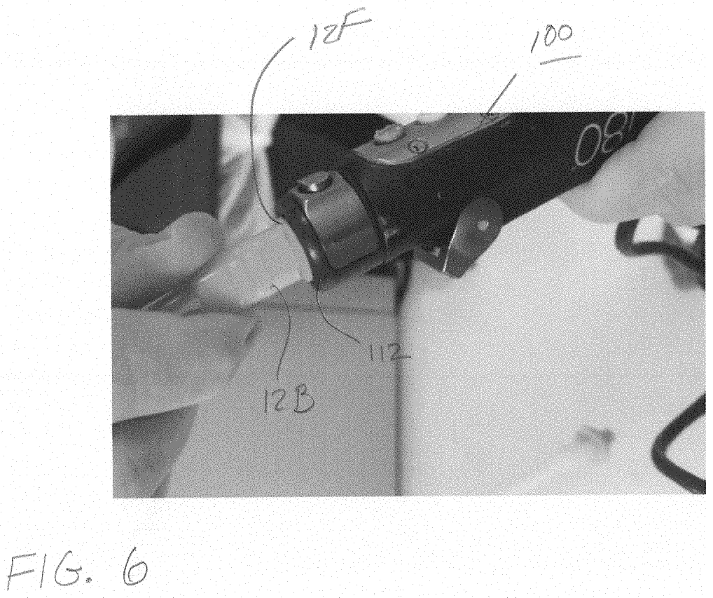

As mentioned above, cleaning adaptor 12 is particularly formed to include the same alignment features as the various surgical “tools” that are attached to the handpiece during use (e.g., burs, drill bits, and the like). Reference is made to FIG. 5, which illustrates a portion of an exemplary conventional handpiece 100 and a pair of notches 110 that are formed on an end termination 112 of handpiece 100. In accordance with the principles of the present invention, cleaning adaptor 12 is formed to include a pair tabs 19 that will align with (and thereafter engage with) notches 110. The view of FIG. 5 shows an intermediate step in the attachment process, with tabs 19 being aligned with notches 110.

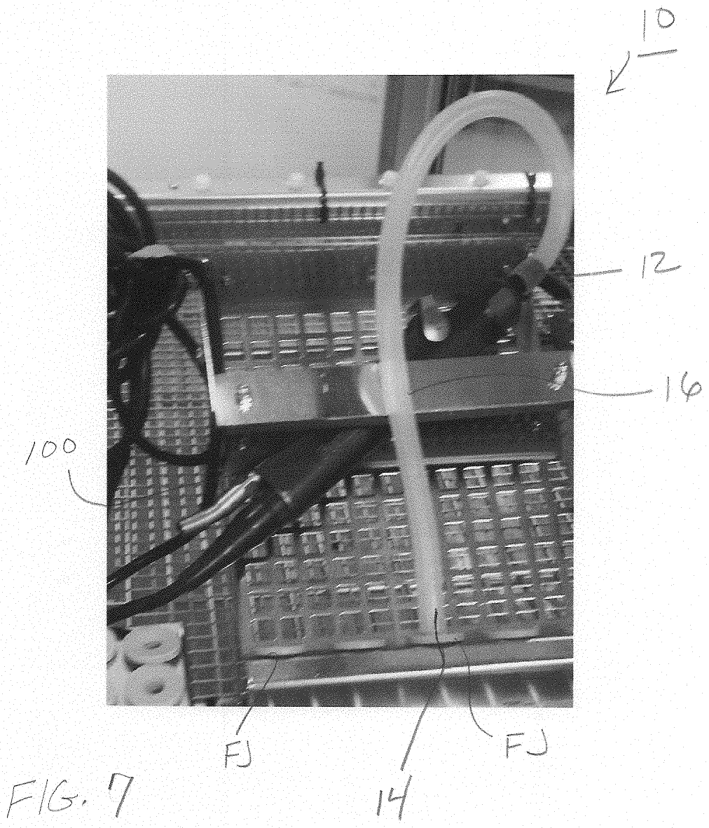

FIG. 6 is a view showing cleaning adaptor 12 as fully engaged with handpiece 100, with first section 12A fully inserted within the handpiece 100. In this case, cleaning adaptor 12 includes a flange interface 12F between sections 12A and 12B, which is shown as creating a physical stop with end portion 112 of handpiece 100.

FIG. 7 is a view of handpiece 100 as loaded within a mechanical washer 200, with directed flow apparatus 10 shown as coupled between an inlet fluid jet FJ of mechanical washer 200 and handpiece 100. In the particular configuration shown in FIG. 7, mechanical washer 200 includes a washer rack system 220 that is specifically designed for attaching auxiliary tubing at inlet fluid port 210.

Cleaning adaptor 12 remains in place within handpiece 100 throughout the multiple cleaning and drying cycles performed by mechanical washer 200, providing the direct flow of high temperature water, detergents, and enzymatic solutions through the irrigation and suction channels of handpiece 100. Once the instruments are unloaded from mechanical washer 200, cleaning adaptor 12 is removed from handpiece 100, with each component of directed flow apparatus 10 then inspected for physical integrity and stored until its next use.

It is to be understood that various modifications and changes in form and detail may be made to the embodiments of the present invention as described above without departing from the spirit and scope of this disclosure. Therefore, the above description should not be construed as limiting the invention but merely as illustrative of possible embodiments. Those skilled in the art will envision other modifications within the scope and spirit of the present invention as defined by the claims appended hereto.

What is claimed is:

1. An apparatus for directing cleaning fluid through a medical instrumentation handpiece during a mechanical washing procedure, the apparatus comprising:
a cleaning adaptor configured to couple to a distal opening of a handpiece, the cleaning adaptor including a central opening for directing movement of cleaning fluid through an interior portion of the handpiece;
an inlet port connector configured to attach to a fluid jet outlet of a mechanical washer; and
a tubing element connected between the inlet port and the cleaning adaptor, providing a path for cleaning fluid exiting the mechanical washer fluid jet outlet to be directed into an interior portion of the handpiece.

2. The apparatus as defined in claim 1 wherein the cleaning adaptor comprises
a first section configured to engage with the handpiece; and
a second section contiguous with the first section, an opposing end of the second section inwardly tapered for creating a water tight fitting with the tubing element.

3. The apparatus as defined in claim 2 wherein the second section of the cleaning adaptor comprises a fluted outer surface around the inwardly tapered region to strengthen the coupled between the tubing element and the cleaning adaptor.

4. The apparatus as defined in claim 2 wherein the first section of the cleaning adaptor includes at least one alignment tab for mating within a handpiece alignment feature to ensure a fixed attachment thereto.

5. The apparatus as defined in claim 2 wherein the first section of the cleaning adaptor is inserted within the handpiece to provide the engagement therewith.

6. The apparatus as defined in claim 5 wherein the cleaning adaptor includes a flange disposed to surround the interface between the first section and the second section, preventing insertion of the second section into the handpiece.

7. The apparatus as defined in claim 1 wherein the inlet port connector comprises a barbed outer surface for providing a watertight connection with the tubing element.
